SEQUENCE LISTING (1 sequence)

SEQ ID NO: 1 - protein, 3256 aa, Homo sapiens
MWPTRRLVTIKRSGVDGPHFPLSLSTCLFGRGIECDIRIQLPVVSKQHCKIEIHEQEAILHNFSSTNPTQVNGSVIDEPVRLKHGDVITIIDRSFRYENESLQNGRKSTEFPRKIREQEPARRVSRSSFSSDPDEKAQDSKAYSKITEGKVSGNPQVHIKNVKEDSTADDSKDSVAQGTTNVHSSEHAGRNGRNAADPISGDFKEISSVKLVSRYGELKSVPTTQCLDNSKKNESPFWKLYESVKKELDVKSQKENVLQYCRKSGLQTDYATEKESADGLQGETQLLVSRKSRPKSGGSGHAVAEPASPEQELDQNKGKGRDVESVQTPSKAVGASFPLYEPAKMKTPVQYSQQQNSPQKHKNKDLYTTGRRESVNLGKSEGFKAGDKTLTPRKLSTRNRTPAKVEDAADSATKPENLSSKTRGSIPTDVEVLPTETEIHNEPFLTLWLTQVERKIQKDSLSKPEKLGTTAGQMCSGLPGLSSVDINNFGDSINESEGIPLKRRRVSFGGHLRPELFDENLPPNTPLKRGEAPTKRKSLVMHTPPVLKKIIKEQPQPSGKQESGSEIHVEVKAQSLVISPPAPSPRKTPVASDQRRRSCKTAPASSSKSQTEVPKRGGRKSGNLPSKRVSISRSQHDILQMICSKRRSGASEANLIVAKSWADVVKLGAKQTQTKVIKHGPQRSMNKRQRRPATPKKPVGEVHSQFSTGHANSPCTIIIGKAHTEKVHVPARPYRVLNNFISNQKMDFKEDLSGIAEMFKTPVKEQPQLTSTCHIAISNSENLLGKQFQGTDSGEEPLLPTSESFGGNVFFSAQNAAKQPSDKCSASPPLRRQCIRENGNVAKTPRNTYKMTSLETKTSDTETEPSKTVSTANRSGRSTEFRNIQKLPVESKSEETNTEIVECILKRGQKATLLQQRREGEMKEIERPFETYKENIELKENDEKMKAMKRSRTWGQKCAPMSDLTDLKSLPDTELMKDTARGQNLLQTQDHAKAPKSEKGKITKMPCQSLQPEPINTPTHTKQQLKASLGKVGVKEELLAVGKFTRTSGETTHTHREPAGDGKSIRTFKESPKQILDPAARVTGMKKWPRTPKEEAQSLEDLAGFKELFQTPGPSEESMTDEKTTKIACKSPPPESVDTPTSTKQWPKRSLRKADVEEEFLALRKLTPSAGKAMLTPKPAGGDEKDIKAFMGTPVQKLDLAGTLPGSKRQLQTPKEKAQALEDLAGFKELFQTPGHTEELVAAGKTTKIPCDSPQSDPVDTPTSTKQRPKRSIRKADVEGELLACRNLMPSAGKAMHTPKPSVGEEKDIIIFVGTPVQKLDLTENLTGSKRRPQTPKEEAQALEDLTGFKELFQTPGHTEEAVAAGKTTKMPCESSPPESADTPTSTRRQPKTPLEKRDVQKELSALKKLTQTSGETTHTDKVPGGEDKSINAFRETAKQKLDPAASVTGSKRHPKTKEKAQPLEDLAGLKELFQTPVCTDKPTTHEKTTKIACRSQPDPVDTPTSSKPQSKRSLRKVDVEEEFFALRKRTPSAGKAMHTPKPAVSGEKNIYAFMGTPVQKLDLTENLTGSKRRLQTPKEKAQALEDLAGFKELFQTRGHTEESMTNDKTAKVACKSSQPDPDKNPASSKRRLKTSLGKVGVKEELLAVGKLTQTSGETTHTHTEPTGDGKSMKAFMESPKQILDSAASLTGSKRQLRTPKGKSEVPEDLAGFIELFQTPSHTKESMTNEKTTKVSYRASQPDLVDTPTSSKPQPKRSLRKADTEEEFLAFRKQTPSAGKAMHTPKPAVGEEKDINTFLGTPVQKLDQPGNLPGSNRRLQTRKEKAQALEELTGFRELFQTPCTDNPTTDEKTTKKILCKSPQSDPADTPTNTKQRPKRSLKKADVEEEFLAFRKLTPSAGKAMHTPKAAVGEEKDINTFVGTPVEKLDLLGNLPGSKRRPQTPKEKAKALEDLAGFKELFQTPGHTEESMTDDKITEVSCKSPQPDPVKTPTSSKQRLKISLGKVGVKEEVLPVGKLTQTSGKTTQTHRETAGDGKSIKAFKESAKQMLDPANYGTGMERWPRTPKEEAQSLEDLAGFKELFQTPDHTEESTTDDKTTKIACKSPPPESMDTPTSTRRRPKTPLGKRDIVEELSALKQLTQTTHTDKVPGDEDKGINVFRETAKQKLDPAASVTGSKRQPRTPKGKAQPLEDLAGLKELFQTPICTDKPTTHEKTTKIACRSPQPDPVGTPTIFKPQSKRSLRKADVEEESLALRKRTPSVGKAMDTPKPAGGDEKDMKAFMGTPVQKLDLPGNLPGSKRWPQTPKEKAQALEDLAGFKELFQTPGTDKPTTDEKTTKIACKSPQPDPVDTPASTKQRPKRNLRKADVEEEFLALRKRTPSAGKAMDTPKPAVSDEKNINTFVETPVQKLDLLGNLPGSKRQPQTPKEKAEALEDLVGFKELFQTPGHTEESMTDDKITEVSCKSPQPESFKTSRSSKQRLKIPLVKVDMKEEPLAVSKLTRTSGETTQTHTEPTGDSKSIKAFKESPKQILDPAASVTGSRRQLRTRKEKARALEDLVDFKELFSAPGHTEESMTIDKNTKIPCKSPPPELTDTATSTKRCPKTRPRKEVKEELSAVERLTQTSGQSTHTHKEPASGDEGIKVLKQRAKKKPNPVEEEPSRRRPRAPKEKAQPLEDLAGFTELSETSGHTQESLTAGKATKIPCESPPLEVVDTTASTKRHLRTRVQKVQVKEEPSAVKFTQTSGETTDADKEPAGEDKGIKALKESAKQTPAPAASVTGSRRRPRAPRESAQAIEDLAGFKDPAAGHTEESMTDDKTTKIPCKSSPELEDTATSSKRRPRTRAQKVEVKEELLAVGKLTQTSGETTHTDKEPVGEGKGTKAFKQPAKRKLDAEDVIGSRRQPRAPKEKAQPLEDLASFQELSQTPGHTEELANGAADSFTSAPKQTPDSGKPLKISRRVLRAPKVEPVGDVVSTRDPVKSQSKSNTSLPPLPFKRGGGKDGSVTGTKRLRCMPAPEEIVEELPASKKQRVAPRARGKSSEPVVIMKRSLRTSAKRIEPAEELNSNDMKTNKEEHKLQDSVPENKGISLRSRRQNKTEAEQQITEVFVLAERIEINRNEKKPMKTSPEMDIQNPDDGARKPIPRDKVTENKRCLRSARQNESSQPKVAEESGGQKSAKVLMQNQKGKGEAGNSDSMCLRSRKTKSQPAASTLESKSVQRVTRSVKRCAENPKKAEDNVCVKKIRTRSHRDSEDI

(19) United States
(12) Patent Application Publication
(10) Pub. No.: US 2026/0328770 A1
(43) Pub. Date: Oct. 22, 2026

(54) CANCER DIAGNOSIS USING KI-67

(71) Applicant: Quest Diagnostics Investments LLC, Secaucus, NJ (US)

(72) Inventors: Jean Marie BRUEY, Encinitas, CA (US); Maher ALBITAR, Coto de Caza, CA (US)

(21) Appl. No.: 19/722,029

(22) Filed: Jun. 26, 2026

Related U.S. Application Data
(63) Continuation of application No. 16/736,325, filed on Jan. 7, 2020, now Pat. No. 12,699,095.
Application No. 16/736,325 is a continuation of application No. 12/610,201, filed on Oct. 30, 2009, now abandoned.
(60) Provisional application No. 61/110,942, filed on Nov. 3, 2008.

(51) Int. Cl. G01N 33/575; G01N 33/57505

(52) U.S. Cl. CPC G01N 33/575; G01N 33/57505; G01N 2800/52; G01N 2800/56

(57) ABSTRACT

The present invention provides methods and compositions for detection, diagnosis, prognosis, monitoring treatment and monitoring progression of cancer by detecting the level of Ki-67 protein in an individual.

### CROSS-REFERENCE TO RELATED APPLICATIONS

This application is a Continuation of U.S. application Ser. No. 16/736,325, filed Jan. 7, 2020, which is a Continuation of U.S. application Ser. No. 12/610,201, filed Oct. 30, 2009, which claims the benefit of U.S. Provisional Applications 61/110,942, filed Nov. 3, 2008, which is hereby incorporated by reference in its entirety.

### SEQUENCE LISTING

The instant application contains a Sequence Listing which has been submitted electronically in XML format and is hereby incorporated by reference in its entirety. Said XML copy, created on Jun. 24, 2026, is named J5363-19204_SL.xml, and is 5,219 bytes in size.

### FIELD OF THE INVENTION

Provided are methods and compositions related to the field of disease detection and, more specifically, for determining a diagnosis and/or prognosis of patients having hyperproliferative disorders.

### BACKGROUND OF THE INVENTION

The following description is provided to assist the reader in understanding the invention and is not admitted to describe or constitute prior art to the invention.

Human Ki-67 protein is expressed in nuclei of proliferating cells in all active phases of the cell cycle, i.e. in G1, S, G2 and mitosis, but not in quiescent G0 cells (Gerdes et al., J. Immunol. 133:1710-1715 (1984)). The cDNA of human Ki-67 and of the murine equivalent are known and show no significant homologies to other proteins (Schluter et al., J. Cell Biol. 123:513-522 (1993); Starborg et al., J. Cell Sci. 109:143-153 (1996)). Human Ki-67 protein has several nuclear localization signals and can physiologically be detected in the cell nucleus, except during mitosis (Heyden et al., Eur. J. Cell Biol. 42:33 (1996)).

Acute lymphoblastic leukemia (ALL) is a form of leukemia (cancer of white blood cells) in which immature white blood cells rapidly multiply and crowd out normal cells in bone marrow. These immature cells do not develop into lymphocytes, which the body uses to fight infection. The immature white blood cells can spread throughout the body and adversely affect other organs. There are about 4,000 new cases of ALL in the United States each year. The earlier acute lymphoblastic leukemia is detected, the more effective the treatment.

White, et al., detects Ki-67 protein in bone marrow cells (J. Clin. Pathol 47 (3): 209-13 (1994)). Jaroslav, et al., detect Ki-67 protein in chronic myelogenous leukemia (CML), ALL, acute myeloid leukemia (AML) and chronic lymphocytic leukemia (CLL) cells (Leuk Lymphoma 46 (11): 1605-1612 (2005)). Xu, et al., detect Ki-67 protein by immunohistochemical staining in cells of children and adults with ALL (Zhonggou Shi Yan Xue Ye Xue Za Zhi 14 (6): 887-890 (2006)). Rykova, E., et al., describes the detection of Ki-67 RNA in plasma and cell bound fraction of patients with breast cancer (Ann. NY Acad. Sci. 1075:328-333 (2006)). U.S. Patent Application No. 2008/0026394 describes electrochemical detection of cancer markers.

### SUMMARY OF THE INVENTION

The present invention provides methods and compositions for detection, diagnosis, prognosis, monitoring treatment and monitoring progression of cancer (e.g., a leukemia or lymphoma) in an individual by assessing Ki-67 protein in an acellular body fluid sample. As described herein, plasma Ki-67 reflects the entire body and not just the site of biopsy so provides information on tumor load, and/or provide more reproducible results.

In one aspect, the invention provides a method for diagnosing an individual as having leukemia or lymphoma by determining the level of Ki-67 protein in an acellular body fluid sample (e.g., serum or plasma) from the individual; comparing the measured level of Ki-67 protein from the individual to a pre-determined cutoff value, and identifying the individual as having leukemia or lymphoma when the Ki-67 protein level of the individual is higher than the cutoff value. Suitable cutoff levels may be determined based on the particular acellular body fluid sample assessed and the particular analytical technique used. Generally, suitable cutoff levels for diagnosis when using plasma are about 900-1500 U Ki-67/ml (e.g., about 1000, 1100, 1200, 1300, or 1400 U/ml). Optionally, the level of β2-microglobulin is also measured, wherein an elevated level compared to disease-free individuals is further diagnostic of leukemia or lymphoma.

The invention also provides a method for diagnosing an individual as having leukemia or lymphoma by measuring the level of Ki-67 protein in an acellular body fluid sample from the individual; calculating a Ki-67 index by normalizing the measured amount of Ki-67 protein to be expressed on a per cell (i.e., per lymphocyte) basis; comparing the Ki-67 index to a pre-determined cutoff value, and identifying the individual as having leukemia when the Ki-67 protein index is higher than said cutoff value. Suitable cutoff values are about 1.5-2.25 U/1000 lymphocytes (e.g., about 1.75, 1.88, 1.95, 2.0, 2.1, 2.2 U/1000 lymphocytes). In this embodiment, the Ki-67 index is calculated by dividing the concentration of Ki-67 (i.e., U/μl) by the concentration of lymphocytes in the same volume. The index may be expressed, for example, as units of Ki-67 per 1000 lymphocytes.

The invention also provides a method for determining the prognosis of an individual with leukemia by measuring the level of Ki-67 protein in an acellular body fluid sample from the individual; calculating a Ki-67 index (e.g., by normalizing the measured amount of Ki-67 protein to the concentration of lymphocytes); comparing the Ki-67 index to a pre-determined cutoff value, and identifying the individual as having a poor prognosis when the Ki-67 protein index is higher than said cutoff value. The prognosis for the individual may be expressed as survival time, complete remission, or remission duration. The blood parameter used to normal the amount of Ki-67 may be the number of total lymphocytes or the number of lymphocytes per unit volume of the blood sample. Suitable cutoff values are about 1.5-2.25 U/1000 lymphocytes/μl (e.g., about 1.75, 1.88, 1.95, 2.0, 2.1, 2.2 U/1000 lymphocytes).

The invention also provides a method for modifying the dosage of chemotherapy administered to an individual diagnosed as having leukemia or lymphoma by determining a Ki-67 protein level index in a first acellular body fluid sample from the individual; determining the Ki-67 protein index in a second acellular body fluid sample from the individual, wherein said second sample is obtained at a later time than said first sample, wherein chemotherapy is administered between obtaining the first and second sample; and modifying said dosage of chemotherapy based on the difference in the Ki-67 protein index in the second sample relative to the first sample, wherein the dosage of chemotherapy in increased when the Ki-67 protein index is higher in the second sample compared to the first sample or reducing or maintaining the dosage of chemotherapy when the Ki-67 protein index is lower in the second sample compared to the first sample. The Ki-67 protein index may be the concentration of Ki-67 protein in the acellular body fluid, or it may be the concentration of Ki-67 protein in the acellular body fluid normalized to the concentration of lymphocytes per unit volume of the acellular body fluid. In one embodiment, the individual is diagnosed as having CML in the chronic phase. In other embodiments, the chemotherapy is Chlorambucil, Cytoxan, or Fludarabine.

In any of the inventive methods, the leukemia or lymphoma may be non-Hodgkin's lymphoma (NHL), acute lymphoblastic leukemia (ALL), Burkitt's lymphoma, acute myeloid leukemia (AML), acute undifferentiated leukemia (AUL), chronic lymphocytic leukemia (CLL), or chronic myeloid leukemia (CML).

One of skill in the art would readily recognize that the measurement of Ki-67 protein can be accomplished using various types of assays well-known in the art. In preferred embodiments, Ki-67 protein is detected using a specific binding agent, preferably an antibody. In another embodiment, the assay is an immunoassay such as an enzyme-linked immunosorbent assay (ELISA) or sandwich-type ELISA. In another embodiment, the assay can be flow cytometry. In the later case, a sandwich-type assay involving capture of an antibody-antigen complex on a bead or microparticle and binding of a labeled second antibody can provide useful assay materials to be evaluated by flow cytometry. In other embodiments, Ki-67 protein is detected by immunoprecipitation which separates proteins from other molecules in the sample. In other embodiments, the measurement of Ki-67 protein is accomplished by immunoblot. In yet other embodiments, the measurement of Ki-67 protein is accomplished by electrochemiluminescence such as by the Meso Scale Discovery (MSD) system. In other embodiments, the Ki-67 protein is detected using protein microarray platform. In one embodiment, the Ki-67 protein is captured directly on microarray solid surface. In another embodiment, the Ki-67 protein is captured is captured indirectly on the microarray solid surface such as through antibody.

“Ki-67 protein” is a marker associated with cancer, as recognized by specific sets of antibodies, which may be used to identify the cell type, stage of differentiation and activity state of a cell. Ki-67 protein and antibodies for detecting Ki-67 protein are described in for example, White, D. M., et al. 47 J. Clin. Pathol. 209-213 (1994); Schwarzenbach, H., et al., 9 (5) Breast Cancer Research R66 (2007); Lokhorst, H. M., et al., 69 (4) Br. J. Haematology 477-481 (1988); Girino M., et al., 85 (1) Acta Haematology 26-30 (1991); and Genbank Accession No. NM_002417. In certain embodiments, Ki-67 protein is present in the liquid phase of a bodily fluid and which remain in the liquid phase after cells have been removed from the bodily fluid (i.e. an acellular body fluid). Ki-67 protein includes fragments of the native cell Ki-67 protein and/or Ki-67 may be physically associated with other biomolecules in the body fluid.

The term “level” as used herein refers to an amount or a concentration of Ki-67 protein. Typically, the level of Ki-67 protein will be expressed as a concentration, or an absolute amount of Ki-67 protein per volume or weight. The term “elevated levels” refers to levels of Ki-67 protein that are above the range of the reference value. In some embodiments, patients with “high” or “elevated” Ki-67 protein levels have activity levels that are higher than the median activity in a population of patients with that disease. In certain embodiments, “high” or “elevated” Ki-67 protein levels for a patient with a particular disease refers to levels that are above the median values for patients with that disorder and are in the upper 40% of patients with the disorder, or to levels that are in the upper 20% of patients with the disorder, or to levels that are in the upper 10% of patients with the disorder, or to levels that are in the upper 5% of patients with the disorder.

The term “determining the level of Ki-67” as used herein refers to measuring or otherwise assessing the amount of the Ki-67 protein in an acellular body fluid obtained from an individual. The assessment of Ki-67 protein level may be relative (e.g., expressed as greater or less than a reference standard or amount, or merely as the presence or absence of a detectable amount) or absolute (e.g., expressed as a concentration). Optionally, when used for detection, prognosis or monitoring cancer, the level of Ki-67 protein in a sample from an individual is compared to the Ki-67 protein level determined in a corresponding sample from person(s) without cancer, known to suffer from, or known to be at risk of a cancer. When determining an individual's prognosis, the level of Ki-67 protein in a sample from an individual diagnosed with cancer is compared to the Ki-67 level determined in a corresponding sample from individuals diagnosed with the same cancer for which the outcome of the cancer is known. In certain embodiments, a threshold (cut-off) level of Ki-67 protein may be established for a given diagnosis or prognosis, and the level of Ki-67 protein in an individual's sample can simply be compared to the threshold level.

The term “circulating Ki-67 index” as used herein refers to a value obtained by determining the ratio of the level of Ki-67 value per given number of lymphocytes/unit volume of acellular body fluid. In one embodiment, circulating Ki-67 index is determined by obtaining a ratio of the Ki-67 level per 1000 lymphocytes. The circulating Ki-67 index value may be indicative of prognosis of cancer patients. Exemplary cancers include but not limited to CLL, ALL, AML, CML, AUL.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. There are many ways that prognosis can be expressed. For example prognosis can be expressed in terms of complete remission rates (CR), overall survival (OS) which is the amount of time from entry to death, remission duration, which is the amount of time from remission to relapse or death.

The phrase “determining the prognosis” as used herein refers to the process by which the practitioner can predict the course or outcome of a condition in an individual. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of years, on average that a patient can expect to survive as a result of a condition or disease. The prognosis of a patient may be considered as an expression of relativism, with many factors affecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

In one embodiment, prognosis of CLL patients can be expressed based on the Rai staging system. According to Rai staging system, status of CLL patients can be divided into 5 stages 0-IV. Stage 0 indicates there are too many lymphocytes in the blood, but there are usually no other symptoms of leukemia. Lymph nodes and the spleen and liver are not swollen, and the number of red blood cells and platelets is normal. Stage I indicates there are too many lymphocytes in the blood and lymph nodes are swollen (lymphadenopathy). The spleen and liver are not swollen and the number of red blood cells and platelets is normal. Stage II indicates there are too many lymphocytes in the blood, lymph nodes are swollen, and either the liver is swollen (hepatomegaly) or the spleen is swollen (splenomegaly). Stage III indicates there are too many lymphocytes in the blood and too few red blood cells (anemia). Lymph nodes and the liver or spleen may be swollen. Stage IV indicates there are too many lymphocytes in the blood and too few platelets (thrombocytopenia). The lymph nodes, liver, or spleen may be swollen, and there may be too few red blood cells (anemia).

The phrase “specific binding agent” as used herein refers to any agent, molecule, or compound that specifically binds Ki-67 protein or portion thereof. Examples include, but are not limited to, antibodies or antibody fragments, ligands, or receptors. These binding agents could be naturally occurring or synthetic and include modified or recombinant proteins. In preferred embodiments the specific binding agent is an antibody. See e.g., White, D. M., et al. 47 J. Clin. Pathol. 209-213 (1994); Schwarzenbach, H., et al., 9 (5) Breast Cancer Research R66 (2007); Lokhorst, H. M., et al., 69 (4) Br. J. Haematology 477-481 (1988); Girino M., et al., 85 (1) Acta Haematology 26-30 (1991); and Genbank Accession No. NM 002417.

As used herein, an “acellular body fluid” is a fluid sample, obtained from a subject, which is substantially free of cells and include, for example, amniotic fluid, blood, cerebral spinal fluid, lactal duct fluid, lymph, peritoneal fluid, plasma, pleural fluid, saliva, serum, sputum, tears, and urine. Preferably, an acellular bodily fluid contains less than about 1% (w/w) whole cellular material. Plasma and serum are examples of acellular bodily fluids. A sample may include a specimen of natural or synthetic origin.

As used herein “absolute lymphocyte count” refers to total number of lymphocyte cells per unit volume of blood. In one embodiment the unit volume of blood may be microliter. In another embodiment, the unit volume of blood may be milliliter. In yet another embodiment, the unit volume of blood may be deciliter.

A “reference sample” comprises a sample of bodily fluid with a known Ki-67 level. The reference sample may contain a known absolute amount of Ki-67 such as an external standard used in a detection assay. Alternatively, a reference sample may be from known normal or diseased subjects wherein the Ki-67 level is designated as being “normal” or diseased.

The term “label” as used herein, refers to any physical molecule directly or indirectly associated with a specific binding agent or antigen which provides a means for detection for that antibody or antigen. A “detectable label” as used herein refers any moiety used to achieve signal to measure the amount of complex formation between a target and a binding agent. These labels are detectable by spectroscopic, photochemical, biochemical, immunochemical, electromagnetic, radiochemical, or chemical means, such as fluorescence, chemifluoresence, or chemiluminescence, electrochemiluminescence or any other appropriate means. Preferred detectable labels include fluorescent dye molecules or fluorophores.

The term “limit of detection (LOD)” as used herein refers to a the point at which a measured value of an analyte in an assay is larger than the uncertainty associated with it. In one embodiment, LOD is defined as 3 standard deviations (SD) from the zero concentration.

The term “about” as used herein in reference to quantitative measurements or values, refers to the indicated value plus or minus 10%.

### BRIEF DESCRIPTION OF THE FIGURES

FIG. 1 shows the cumulative proportion of ALL patients surviving in two populations of patients: 1) patients having plasma concentration of Ki-67 protein of less than 1,500 U/ml (solid line), and 2) patients having plasma concentration of Ki-67 protein of greater than 1,500 U/ml (dashed line).

FIG. 2 is the amino acid sequence of Ki-67 protein as provided at Genbank Accession No. NP 002408 (SEQ ID NO:1).

FIG. 3 shows a box plot of the levels of circulating Ki-67 protein in plasma in normal individuals (N) and ALL patients as determined by MSD® Electrochemiluminiscent method. The units of Ki-67 levels are expressed as units/ml (U/ml). Asterisks indicate outliers; open circles indicate extreme values. Median values of the levels of Ki-67 protein is represented as a horizontal line within the box in each case.

FIG. 4 shows a standard curve for the Ki-67 assay described in Example 1.

FIG. 5 shows the results of detection of Ki-67 protein in plasma in normal and CML patients by direct Western blotting and by immunoprecipitation followed by Western blotting. Albumin was used as a positive control in direct Western blotting and IgG was used as a negative control for immunoprecipitation by Ki-67 antibody.

FIGS. 6A and 6B show the effect of serum starvation on the expression level of Ki-67 protein in cultured cells. FIG. 6A shows a Western blot analysis of Ki-67 protein expression levels in cultured cells in presence of serum or in serum starved condition. Beta-actin served as a positive control for the assay. FIG. 6B shows the results of the analysis of cell lysates (in presence of serum versus serum starved) for the detection of Ki-67 protein using MSD® ECL method.

FIG. 7 shows a correlation of survival of CLL patients in months with the normalized level of Ki-67 protein (relative to the number of lymphocytes) in plasma. CLL patients were monitored for survival in months from the time of testing the absolute lymphocyte count and the plasma Ki-67 level of the individual. A normalized Ki-67 value is obtained by taking the ratio of the Ki-67 level to the absolute lymphocyte count. The solid line represents individual with a normalized Ki-67 level of less than 1.88 U/1000 lymphocytes. The broken line represents a normalized Ki-67 value of greater than or equal to 1.88 U/1000 lymphocytes.

FIG. 8 shows a correlation of the relative Ki-67 level with the prognosis of CLL patients. Prognosis of CLL patients were classified according to Rai classification from 0-IV.

Plasma Ki-67 values and absolute lymphocyte count were measured of the CLL patients. A normalized Ki-67 value is obtained by taking the ratio of the Ki-67 level to the absolute lymphocyte count. Normalized Ki-67 values were plotted against the Rai-classified CLL patient groups in box plots. The mean normalized Ki-67 for each group is indicated as a small square within each box. The standard deviation of measurement for each group is indicated.

FIG. 9 shows a box plot of the normalized Ki-67 levels in treated and untreated CLL patients. Plasma Ki-67 values and absolute lymphocyte count were measured from CLL patients treated with or without (untreated) Chlorambucil. A normalized Ki-67 value is obtained by taking the ratio of the Ki-67 level to the absolute lymphocyte count. The normalized Ki-67 values were plotted for the treated and untreated groups.

FIG. 10 shows a correlation of survival of CLL patients in months with the normalized level of Ki-67 protein (relative to the number of lymphocytes) in plasma after starting a new therapy. CLL patients were monitored for survival in months from the start of treatment with Cytoxan and Fludarabine. A normalized Ki-67 value is obtained by taking the ratio of the plasma Ki-67 level to the absolute lymphocyte count. The solid line (Group 1) represents the population of individuals with a normalized Ki-67 level of greater than 1.88 U/1000 lymphocytes. The dashed line (Group 2) represents the population of individuals with a normalized Ki-67 value of less than or equal to 1.88 U/1000 lymphocytes. An open circle (∘) “complete” indicates dead and a plus (+) “Censored indicates alive.

FIG. 11 shows a box plot of the levels of circulating Ki-67 protein in plasma in normal individuals (N) and CLL patients as determined by MSD® Electrochemiluminiscent method. The units of Ki-67 levels are expressed as U/ml. Median values of the levels of Ki-67 protein is represented as a horizontal line within the box in each case.

FIG. 12 shows the distribution of normalized Ki-67 values in the plasma of 194 CLL patients. A normalized Ki-67 value is obtained by taking the ratio of the plasma Ki-67 level to the absolute lymphocyte count.

FIG. 13 shows the cumulative proportion of AML patients surviving in two populations of patients: 1) patients having plasma concentration of Ki-67 protein of less than 2100 U/ml (solid line), and 2) patients having plasma concentration of Ki-67 protein of greater than 2100 U/ml (dashed line).

FIG. 14 shows the cumulative proportion of CML patients surviving in two populations of patients: 1) patients having plasma concentration of Ki-67 protein of less than 354 U/ml (solid line), and 2) patients having plasma concentration of Ki-67 protein of greater than 354 U/ml (dashed line).

FIG. 15 shows the cumulative proportion of CML patients surviving in two populations of patients: 1) patients having circulating Ki-67 index of less than 1.20 U/1000 lymphocytes (dashed line), and 2) patients having circulating Ki-67 index of Ki-67 protein of greater than 1.20 U/ml (solid line).

### DETAILED DESCRIPTION OF THE INVENTION

The present inventions described herein are based on the discovery and characterization that Ki-67 protein can be detected in acellular body fluid samples for the prognosis and diagnosis of certain cancers, including various leukemias.

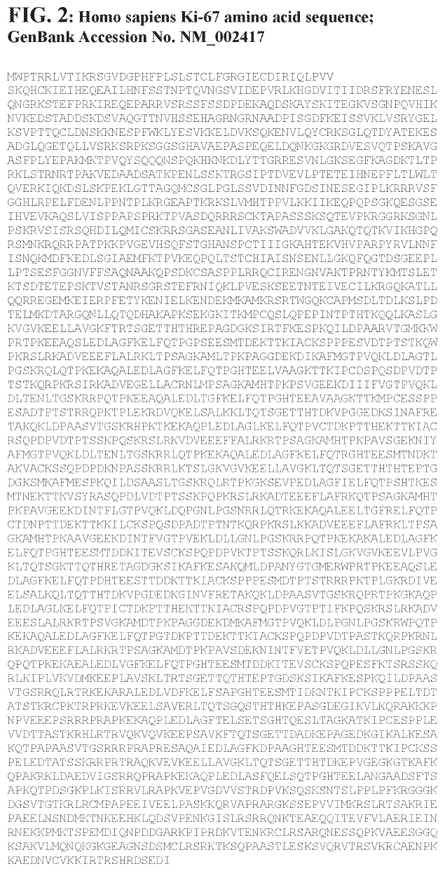

Exemplary DNA sequence of Ki-67 includes but not limited to GenBank accession number NM_002417. Exemplary amino acid sequence of Ki-67 protein includes GenBank accession number NP_002408 and is provided in FIG. 2 (SEQ ID NO: 1).

The present invention provides methods for detecting the level of Ki-67 protein of an individual. The level of Ki-67 protein is determined by assaying a biological sample from an individual for Ki-67 protein. Ki-67 protein is assayed using assays known in the art and binding agents specific to the markers of interest. The levels of Ki-67 protein in the individual are compared to the levels in normal individuals free from any disorder or compared to another individual having particular disorder. Ki-67 protein levels which deviate from the normal levels can be used to diagnose a disorder or to determine the prognosis or treatment for an existing disorder. Further, changes in the Ki-67 protein levels over time can be used to assess progression of the disorder or success of the treatment thereof.

### Plasma or Serum Preparation Methods

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70 degrees centigrade until thawed and used. “Fresh” plasma or serum should be refrigerated or maintained on ice until used, with nucleic acid (e.g., RNA, DNA or total nucleic acid) extraction being performed as soon as possible. Exemplary methods are described below.

Blood can be drawn by standard methods into a collection tube, preferably siliconized glass, either without anticoagulant for preparation of serum, or with EDTA, sodium citrate, heparin, or similar anticoagulants for preparation of plasma. The preferred method if preparing plasma or serum for storage, although not an absolute requirement, is that plasma or serum be first fractionated from whole blood prior to being frozen. This reduces the burden of extraneous intracellular RNA released from lysis of frozen and thawed cells which might reduce the sensitivity of the amplification assay or interfere with the amplification assay through release of inhibitors to PCR such as porphyrins and hematin. “Fresh” plasma or serum may be fractionated from whole blood by centrifugation, using preferably gentle centrifugation at 300-800 times gravity for five to ten minutes, or fractionated by other standard methods. High centrifugation rates capable of fractionating out apoptotic bodies should be avoided.

### Determination of Absolute Lymphocyte Count

Absolute counts are calculated by multiplying the total white blood cell count by the percent of the specific cell type of interest. While percentage reports are considered adequate for most patients, absolute values are more important for patients with hematologic disorders.

If lymphocyte or other counts are reported as percentages of the total white blood count (wbc), the absolute values can be calculated as follows: total wbc x % cell type reported/100. This formula can be used for calculating the absolute lymphocyte count, absolute neutrophil count, etc.

An exemplary reference range for total white blood cell counts in healthy individuals is 4.0-11.0×10³ per microliter. The various types of white blood cells are often expressed as a percentage of the total white blood cell count. Exemplary percentage ranges are as follows:
Basophils—0 to 2%, Eosinophils—0% to 3%, Lymphocytes—25% to 35%, Monocytes—3% to 10%, Neutrophils—50% to 60%.

These percentages are derived from 1) a microscopic examination of blood performed manually in which some hundreds of cells are differentiated from each other (this procedure is called a differential) or 2) a machine scored differentiation based on cell patterns.

By way of example, if the total white count reported is 25,000 and the percentage of lymphocytes reported is 80%, the calculation is as follows: 25,000×80/100. The result is an absolute lymphocyte count of 20,000.

Protein Extraction from Acellular Body Fluids

The Ki-67 protein may be extracted from the acellular body fluid sample by any suitable method including, for example, by immobilization on a solid surface such as microwells, beads or microarray using Ki-67 specific antibodies anchored to the solid surface for detection and quantification.

Plasma purification methods are known in the art such. See e.g., Cohn, E. J., et al, Am. Chem. Soc. 62:3396-3400. (1940); Cohn, E. J., et al., J. Am. Chem. Soc. 72:465-474 (1950); Pennell, R. B. Fractionation and isolation of purified components by precipitation methods. p. 9-50. In The Plasma Proteins, Vol. 1. F. W. Putman (ed.). Academic Press, New York (1960); and U.S. Pat. No. 5,817,765.

### Antibodies to Ki-67 Protein

Methods of generating antibodies are well known in the art, see, e.g., Sambrook, et al., 1989, Molecular Cloning: A Laboratory Manual, Second Edition, Cold Spring Harbor Press, Plainview, NY. Antibodies for Ki-67 are known in the art and are described, for example, in White, D. M., et al. 47 J. Clin. Pathol. 209-213 (1994); Schwarzenbach, H., et al., 9 (5) Breast Cancer Research R66 (2007); Lokhorst, H. M., et al., 69 (4) Br. J. Haematology 477-481 (1988); Girino M., et al., 85 (1) Acta Haematology 26-30 (1991); and Genbank Accession No. NM 002417.

Antibodies may be detectably labeled by methods known in the art. Labels include, but are not limited to, radioisotopes such as ³H, ¹⁴C, ³⁵S, ³²P, ¹²³I, ¹²⁵I, ¹³¹I), enzymes (e.g., peroxidase, alkaline phosphatase, beta-galactosidase, luciferase, alkaline phosphatase, acetylcholinesterase and glucose oxidase), enzyme substrates, luminescent substances (e.g., luminol), fluorescent substances (e.g., FITC, rhodamine, lanthanide phosphors), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags) and colored substances. In binding these labeling agents to the antibody, the maleimide method (Kitagawa, T., et al., 79 J. Biochem. 233-236 (1976)), the activated biotin method (Hofmann, K., et al. 100 J. Am. Chem. Soc. 3585 (1978)) or the hydrophobic bond method, for instance, can be used.

In some embodiments, labels are attached via spacer arms of various lengths to reduce potential steric hindrance. Antibodies may also be coupled to electron dense substances, such as ferritin or colloidal gold, which are readily visualized by electron microscopy.

Where a radioactive label is used as a detectable substance, proteins may be localized by autoradiography. The results of autoradiography may be quantitated by determining the density of particles in the autoradiographs by various optical methods, or by counting the grains.

The antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against Ki-67.

Antibodies to Ki-67 protein are available commercially through multiple sources. For example, purified antibodies directed Ki-67 protein are available labeled or unlabeled through Abcam Inc. (Cambridge, MA).

### Immunoassays to Detect Ki-67 Protein

Immunoassays, or assays to detect an antigen using an antibody, are well known in the art and can take many forms, e.g., radioimmunoassay, immunoprecipitation, Western blotting, enzyme-linked immunosorbent assay (ELISA), electrochemiluminescence assay, and 2-site or sandwich immunoassay.

In preferred embodiments, a sandwich ELISA is used. In this assay, two antibodies to different segments, or epitopes, of the antigen are used. The first antibody (capture antibody) is coupled to a solid support. When a sample of bodily fluid is contacted with the capture antibody on the solid support, the antigen contained in the bodily fluid is captured on the solid support through a specific interaction between antigen and antibody, resulting in the formation of a complex. Washing of the solid support removes unbound or non-specifically bound antigen. Subsequent exposure of the solid support to a detectably-labeled second antibody (detection antibody) to the antigen (generally to a different epitope than the capture antibody) enables the detection of bound or captured antigen. As would be readily recognized by one of skill in the art, assaying additional markers in parallel to assaying for Ki-67 protein is possible with the use of distinct pairs of specific antibodies, each of which is directed against a different marker. The capture and detection antibodies may be individually monoclonal or polyclonal antibodies.

Relative or actual amounts of Ki-67 protein in body fluids can be determined by methods well known in the art. See e.g., Drach, J., et al., 10 (6) Cytometry 743-749 (1989). For example, a standard curve can be obtained in the ELISA using known amounts of Ki-67 protein. The actual amount of the Ki-67 protein in a body fluid may thus be determined using the standard curve. Another approach that does not use a standard curve is to determine the dilution of body fluid that gives a specified amount of signal. The dilution at which 50% of the signal is obtained is often used for this purpose. In this case, the dilution at 50% maximal binding of Ki-67 protein in a patient body fluid is compared with the dilution at 50% of maximal binding for Ki-67 protein obtained in the same assay using a reference sample (i.e., a sample taken from the corresponding bodily fluid of normal individuals, free of proliferative disorders).

Methods of identifying the binding of a specific binding agent to Ki-67 protein are known in the art and vary dependent on the nature of the label. In preferred embodiments, the detectable label is a fluorescent dye. In other embodiments, electrochemiluminescence (“ECL”) assay is used for detection of Ki-67 protein. In one embodiment, this ECL assay employs Meso Scale Discovery (MSD®) technology which is an adaptation of ELISA assays. A capture antibody specific for Ki-67 (mouse anti human Ki-67) is coated onto the wells. Nonspecific binding was first blocked by overnight incubation at 4° C. with Blocker A solution provided by the manufacturer. Samples including standards of known Ki-67 concentrations, specimens and controls are diluted, added to the wells and incubated during 2 hours. Plates are washed 3 times to remove the unbound samples and the detection antibody rabbit anti human Ki-67 is added and incubated. After washing to remove the unbound rabbit anti human Ki-67 antibody, SULFO-TAG anti rabbit antibody is added to the wells. The reading is achieved by adding a MSD® Read Buffer solution which contains Tripropylamine (TPA) to the plate. Each sample is measured in duplicate Standard curves for the estimation of Ki-67 concentration are generated by using serial dilutions of HL60 lysate. The plate is read using MSD Sector Imager 2400 Instrument for electrochemiluminescence RLU (relative light unit) signal. This RLU signal is analyzed and compared with RLU signals of standard to get the concentration of each sample.

### Diagnosis of Cancer

The level of Ki-67 protein in a test sample can be used in conjunction with clinical factors other than Ki-67 protein to diagnose a disease. In these embodiments, the level of Ki-67 protein measured in the test sample is compared to a reference value to determine if the levels of Ki-67 protein is elevated or reduced relative to a reference value. Preferably, the reference value is the Ki-67 protein level measured in a comparable sample from one or more healthy individuals. An increase or decrease in Ki-67 protein may be used alone or in conjunction with clinical factors other than the level of Ki-67 protein to diagnose a disease.

Clinical factors of particular relevance in the diagnosis of cancer include, but are not limited to, the patient's medical history, a physical examination of the patient, complete blood count, examination of bone marrow cells, cytogenetics, and immunophenotyping of blood cells.

Monitoring Progression and/or Treatment

In one aspect of the invention, the level of Ki-67 protein in biological sample of a patient is used to monitor the effectiveness of treatment. In preferred embodiments, the level of a Ki-67 protein in a test sample obtained from a treated patient, can be compared to the level from a reference sample obtained from that patient prior to initiation of the same treatment. Clinical monitoring of treatment preferably entails that each patient serve as his or her own baseline control.

A decrease in Ki-67 protein in the patient test sample as compared to the patient's reference sample is indicative of an in vivo effect of the treatment at the time the test sample was obtained. In some embodiments, test samples are obtained at multiple time points following administration of the treatment. In these embodiments, measurement of Ki-67 protein in the test samples provides an indication of the extent and duration of in vivo effect of the treatment.

In another aspect of the invention, the level of Ki-67 protein relative to the absolute lymphocytes count in biological sample of a patient is used to monitor the effectiveness of treatment. In preferred embodiments, the level of a Ki-67 protein in a test sample obtained from a treated patient, a ratio of the level of Ki-67 protein to the absolute lymphocyte count is obtained and the ratio can be compared to the ratio from a reference sample obtained from that patient prior to initiation of the same treatment. Clinical monitoring of treatment preferably entails that each patient serve as his or her own baseline control.

A decrease in the ratio of Ki-67 protein to the absolute lymphocytes in the patient test sample as compared to the patient's reference sample is indicative of an in vivo effect of the treatment at the time the test sample was obtained. In some embodiments, test samples are obtained at multiple time points following administration of the treatment. In these embodiments, measurement of the ratio Ki-67 protein to the absolute lymphocytes in the test samples provides an indication of the extent and duration of in vivo effect of the treatment.

### Determining Prognosis

Provided herein are methods of using Ki-67 protein level in a test sample from a patient in conjunction with clinical factors in determining the prognosis for a patient having cancer. In some embodiments, prognosis may be a prediction of the likelihood that a patient will survive for a particular period of time, or the prognosis is a prediction of how long a patient may live, or the prognosis is the likelihood that a patent will recover from a disease or disorder. There are many ways that prognosis can be expressed. For example prognosis can be expressed in terms of complete remission rates (CR), overall survival (OS) which is the amount of time from entry to death, remission duration, which is the amount of time from remission to relapse or death.

In certain embodiments high levels of Ki-67 protein are used as indicators of an unfavorable prognosis, for example, in ALL. In another embodiment, high levels of Ki-67 protein are used as indicators of an longer survival time, for example in CML and AML. According to the method, the determination of prognosis can be performed by comparing the measured Ki-67 protein level to levels determined in comparable samples from healthy individuals or to levels known to corresponding with favorable or unfavorable outcomes. The absolute Ki-67 protein levels obtained may depend on a number of factors, including but not limited to the laboratory performing the assays, the assay methods used, the type of body fluid sample used and the type of disease a patient is afflicted with. According to the method, values can be collected from a series of patients with a particular disorder to determine appropriate reference ranges of Ki-67 protein for that disorder. One of ordinary skill in the art is capable of performing a retrospective study that compares the determined Ki-67 protein levels to the observed outcome of the patients and establishing ranges of levels for each activity that can be used to designate the prognosis of the patients with a particular disorder. For example, Ki-67 protein levels in the lowest range would be indicative of a more favorable prognosis, while Ki-67 protein levels in the highest ranges would be indicative of an unfavorable prognosis.

Because the level of Ki-67 protein in a test sample from a patient relates to the prognosis of a patient in a continuous fashion, the determination of prognosis can be performed using statistical analyses to relate the determined activity levels to the prognosis of the patient. A skilled artisan is capable of designing appropriate statistical methods. For example the methods of the present invention may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses.

In some embodiments, a circulating Ki-67 index value is indicative of prognosis such as survival time. A circulating Ki-67 index value may be determined for example, by obtaining a ratio of the Ki-67 protein per 1000 circulating lymphocytes/μl of plasma. In some embodiment, higher circulating Ki-67 index value is indicative of poor prognosis such as in CLL, ALL, AML, CML, AUL.

In certain embodiments, the prognosis of ALL, AML, AUL, CLL or CML patients can be correlated to the clinical outcome of the disease using the level of Ki-67 protein and other clinical factors. Simple algorithms have been described and are readily adapted to this end. The approach by Giles et. al., British Journal of Hemotology, 121:578-585, is exemplary. As in Giles et al., associations between categorical variables (e.g., proteasome activity levels and clinical characteristics) can be assessed via crosstabulation and Fisher's exact test. Unadjusted survival probabilities can be estimated using the method of Kaplan and Meier. The Cox proportional hazards regression model also can be used to assess the ability of patient characteristics (such as proteasome activity levels) to predict survival, with ‘goodness of fit’ assessed by the Grambsch-Therneau test, Schoenfeld residual plots, martingale residual plots and likelihood ratio statistics (see Grambsch, 1995; Grambsch et al, 1995).

In some embodiments of the invention, multiple prognostic factors, including Ki-67 protein level, are considered when determining the prognosis of a patient. For example, the prognosis of an AML or ALL patient may be determined based on Ki-67 protein and one or more prognostic factors selected from the group consisting of cytogenetics, performance status, AHD (antecedent hematological disease), age, and diagnosis (e.g., MDS v. AML). In certain embodiments, other prognostic factors may be combined with the Ki-67 protein level in the algorithm to determine prognosis with greater accuracy.

In some embodiments, a ratio of the level of Ki-67 protein in acellular body fluid to the absolute lymphocyte count is indicative of prognosis of certain cancers including breast cancer, prostate cancer, lymphoma, and leukemia. The ratio is compared to a cutoff value or a reference value. A ratio of the level of Ki-67 protein in acellular body fluid to the absolute lymphocyte count lower than the cutoff value or a reference value is indicative of better prognosis as compared to a ratio higher than the cutoff or reference value.

### Example 1

### Reproducibility of the Assay for Detection of Ki-67 Protein by MSD® Electrochemiluminiscent Method

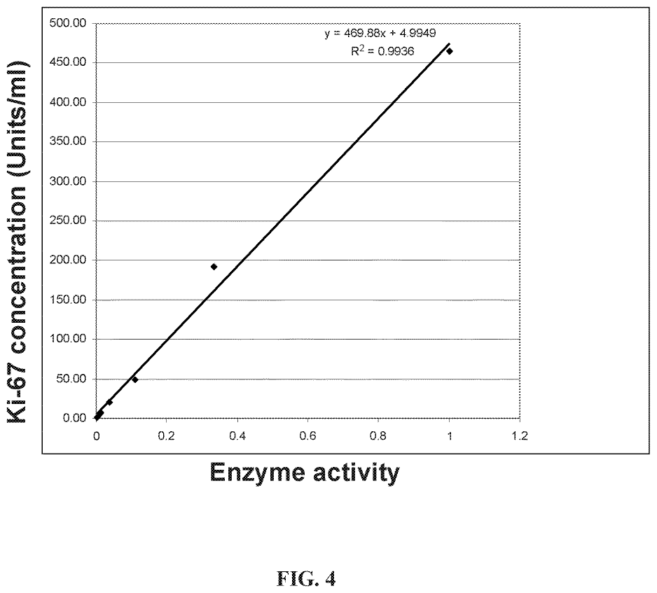

Standard Curve Reproducibility: Seven standards containing Ki-67 at various concentrations (500, 166.66, 55.55, 18.51, 6.17, 2.15, and 0.68 Units/ml) was run in a daily assay set up to generate a calibration curve. The slope of the curve was used as a conversion factor to calculate enzyme activity. The Ki-67 standard curve showed good linearity (R2) and reproducibility (CV %) (FIG. 4). The standard curve was run on various days over several months and showed excellent reproducibility.

Intra-Assay Variation: The intra-assay variation is defined as the reproducibility of a sample within an assay. Plasma controls (high and low control) involved 8 replicates to evaluate reproducibility within runs (Table 1). The terms “low,” “medium” and “high” refer to relative levels of Ki-67 protein in the plasma samples. The intra-assay CV % for plasma Ki-67 was 5.22% for low normal, 5.56% for low patient and 1.54% for high patient.

TABLE 1
Intra-assay Variation and accuracy

| | Patient | | | Normal | | |
|---|---|---|---|---|---|---|
| repeat | low | med | high | low | low | high |
| 1 | 37061 | 171567 | 485095 | 57460 | 5991 | 74363 |
| 2 | 43240 | 174448 | 478073 | 62089 | 6650 | 55567 |
| 3 | 39913 | 167472 | 479059 | 60579 | 6474 | 61129 |
| 4 | 41812 | 161963 | 478472 | 59274 | 6157 | 47193 |
| 5 | 42662 | 161212 | 477979 | 56252 | 5996 | 54721 |
| 6 | 42040 | 158579 | 463003 | 57931 | 5800 | 45820 |
| Mean | 41121.33 | 165873.5 | 476946.8 | 58930.83 | 6178 | 56465.5 |
| Ki-67 | | | | | | |
| STDEV | 2286.034 | 6304.356 | 7347.709 | 2151.623 | 323.0536 | 10442.46 |
| % CV | 5.55924 | 3.800701 | 1.540572 | 3.651098 | 5.229096 | 18.49353 |

Inter-Assay Variation: The inter-assay variation is defined as the reproducibility of a sample between assays. Five plasma controls (1 high, 2 med, and 2 low Ki-67 levels) were run 6 different days to determine inter-day assay reproducibility (Table 2). The Ki-67 assay has a CV % of less than 15 for the inter assay variation. The CV % for low, medium and high plasma concentration of Ki-67 protein was 4.11% (average of 4.97 and 3.25), 5.75% (average of 6.84 and 4.67) and 9.46%, respectively.

TABLE 2
Inter-Assay Variation and Accuracy

| | Patient 1 | Patient 2 | Patient 3 | Patient 4 | Patient 5 |
|---|---|---|---|---|---|
| dates | high | med | med | low | low |
| Day 1 | 124986 | 68704 | 46657 | 3826 | 2631 |
| Day 2 | 121214 | 65010 | 42044 | 3493 | 2847 |
| Day 6 | 110414 | 65642 | 41696 | 3759 | 2626 |
| Day 8 | 116377 | 68829 | 43876 | 3356 | 2785 |
| Day 10 | 104208 | 63735 | 45903 | 3576 | 2676 |
| Day 14 | 96819 | 56740 | 45398 | 3487 | 2734 |
| Mean Ki-67 | 112336.3 | 64776.67 | 44262.33 | 3582.833 | 2716.5 |
| SD | 10636.79 | 4434.449 | 2067.476 | 178.28 | 88.50932 |
| CV | 9.468702 | 6.84575 | 4.670959 | 4.975951 | 3.258212 |

Selectivity is the ability of an analytical method to differentiate and quantify the analyte in the presence of other components in the sample. For selectivity, analyses of blank samples of the appropriate biological matrix were obtained, tested for interference and selectivity ensured at the lower limit of quantification. The Ki-67 Zero standard was run in 40 replicates each and the selectivity of the assay for detection of Ki-67 by MSD® Electrochemiluminiscent assay was found to be 2996.3 RLU. The limit of detection (LOD) is defined as the concentration of an analyte required to give a signal equal to the background (blank) plus three times the standard deviation of the blank.

### Example 2

### Serum Starvation Downregulates Ki-67 Protein Expression in Cultured Cells

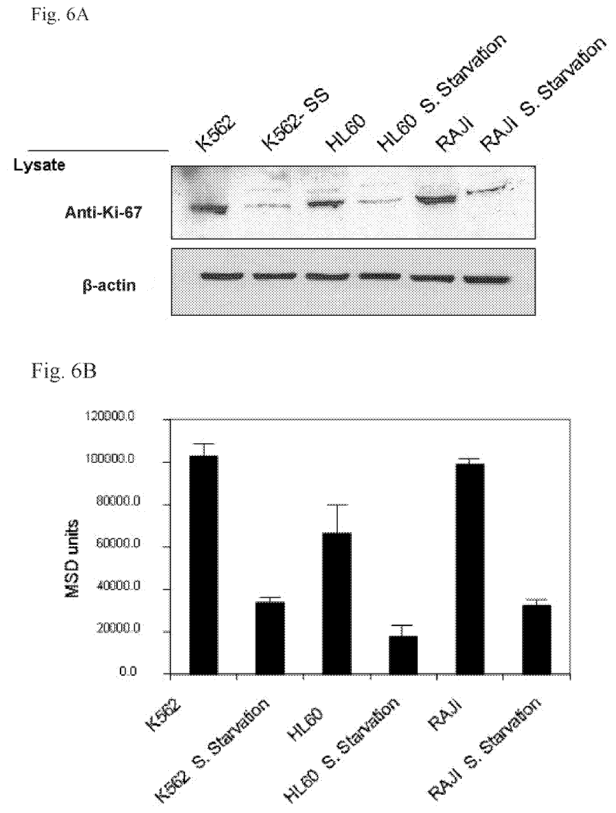

K562, HL60, and Raji cells were cultured in media containing 10% FBS or 0.2% FBS. Cell lysates (100 μg) were prepared and analyzed by SDS-PAGE/immunoblotting using anti-Ki-67 or anti-actin antibodies. Antibody detection was accomplished by ECL, with exposure to x-ray film. Ki-67 protein is downregulated under serum starved condition as shown in FIG. 6A.

Similar results were obtained when the lysates were also analyzed using MSD® ECL method. Ki-67 protein is downregulated under serum starved condition and shown in FIG. 6B.

### Example 3

### Detection of Ki-67 Protein in the Plasma of CLL Patients

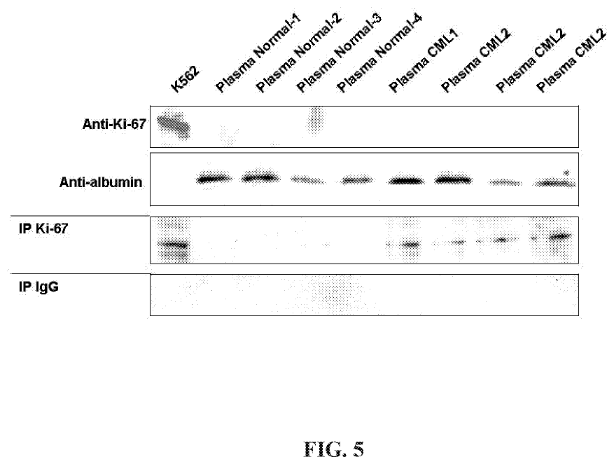

Plasma samples were obtained from normal individuals and chronic myelogenous leukemia (CML) patients. The detection of Ki-67 protein in plasma was evaluated by Western blotting (with or without prior immunoprecipitation) with anti-Ki-67 antibody (mouse monoclonal anti-Ki-67 antibody from Invitrogen™, Clone 7B11; and rabbit polyclonal anti-Ki-67 antibody from Santa Cruz Biotechnology, Inc., Clone H300). When Western blotting was performed after immunoprecipitation, the immunoprecipitate was diluted 1:1 in lysis buffer prior to Western blotting. The top row of results in FIG. 5 shows that Ki-67 was not detectable directly in any normal or patient plasma without pre-immunoprecipitation. The second row from the top in FIG. 5 shows that albumin was directly detectable in each plasma sample analyzed.

The bottom two rows in FIG. 5 depict immunopreciptation of Ki-67 followed by Western blotting using anti-Ki-67 antibody. The results in the third row from the top in FIG. 5 shows that Ki-67 was detectable in CML patient plasma but not in normal plasma.

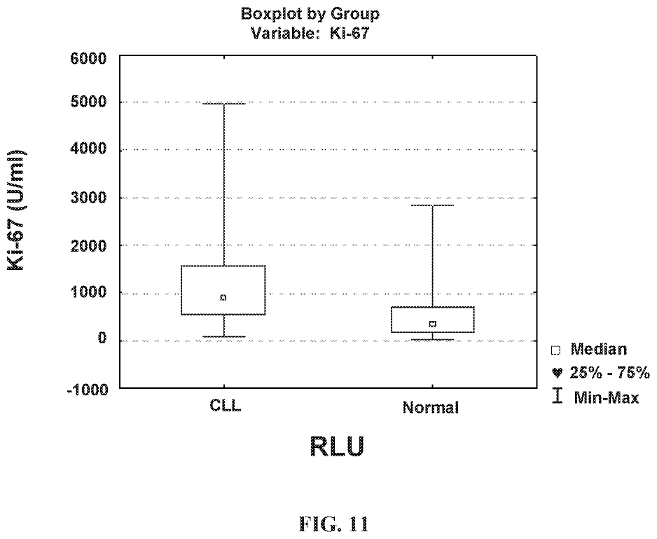

Following qualitative detection of plasma Ki-67 in CLL patients, the plasma Ki-67 protein was quantified using Meso Scale Discovery (MSD) in the plasma of patients with CLL (n=194) and normal patients (n=96). Ki-67 protein levels were significantly higher in patients with CLL and shown in FIG. 11.

### Example 4

### Correlation of Circulating Ki-67 Levels with Other Clinical Markers in CLL Patients

The absolute plasma Ki-67 levels in CLL patients were correlated to other hematological parameters and CLL markers using the Kruskal-Wallis test and Spearman Rank. As shown in Table 4, there was little or no correlation of plasma Ki-67 protein levels in CLL patients with any of the markers.

TABLE 31
Patient Characteristics.

| Characteristic | % | |
|---|---|---|
| Male | 68 | |
| Rai III-IV | 25 | |
| Splenomegaly | 28 | |
| Hepatomegaly | 4 | |
| Lymph nodes | 65 | |
| | Median (range) | |
| Age | 61 | (34-84) |
| WBC (×10³/uL) | 21.25 | (1.4-321) |
| HGB (g/dL) | 13 | (3.3-16.8) |
| B2M (mg/L) | 3.1 | (1.4-18.1) |
| Platelets (×10⁶/L) | 177 | (4-511) |

TABLE 4
Correlation of circulating absolute Ki-67
levels with various clinical parameters

| Variable | Valid-N | Spearman-R | t(N − 2) | P |
|---|---|---|---|---|
| HGB (g/dL) | 194 | 0.047 | 0.654 | 0.51 |
| PLT (×10⁶/L) | 194 | 0.100 | 1.399 | 0.16 |
| WBC (×10⁶/L) | 194 | 0.021 | 0.287 | 0.77 |
| Lymphocytes (%) | 194 | −0.047 | −0.649 | 0.51 |
| Liver enlargement (cm) | 192 | 0.055 | 0.766 | 0.44 |
| Spleen enlargement (cm) | 187 | 0.023 | 0.314 | 0.75 |
| Lymph node sites- | 190 | −0.059 | 0.815 | 0.42 |
| enlarged | | | | |
| B2M (mg/L) | 194 | 0.024 | 0.336 | 0.74 |
| BM Cellularity (%) | 189 | 0.118 | 1.624 | 0.11 |
| BM-Lymphocytes (%) | 194 | 0.079 | 1.099 | 0.27 |
| RAI Stage | | 0.034 | 0.4667 | 0.64 |
| Albumin | | 0.026 | 0.364 | 0.72 |
| Creatinine (mg/dL) | 193 | −0.091 | −1.265 | 0.21 |
| IgG (mg/dL) | 184 | 0.038 | 0.519 | 0.60 |
| IgA (mg/dL) | 183 | −0.079 | −1.073 | 0.28 |
| IgM (mg/dlL | 184 | 0.010 | 0.134 | 0.89 |
| Total Protein (g/dL) | 58 | −0.202 | −1.541 | 0.13 |
| CD11/CD22 (%) | 194 | −0.036 | −0.501 | 0.62 |
| CD11C (%) | 194 | −0.071 | −0.990 | 0.32 |
| CD22 (%) | 194 | 0.019 | 0.262 | 0.79 |

Abbreviations:
HGB: hemoglobin;
PLT: platelet count;
WBC: white blood cell count;
B2M: β2 microglobulin;
BM: Bone Marrow;
Ig, immunoglobulin.

### Example 5

### Correlation of Circulating Ki-67 Index Values with the Survival of CLL Patients

The hematological parameters and CLL markers measured in Example 5 were normalized to the number of circulating lymphocytes in order to obtain a proliferation fraction. This normalization ensures that the Ki-67 levels reflect leukemic proliferation rather than disease volume. These normalized results are referred to as the Ki-67 index. Specifically, the Ki-67 index value is obtained by measuring the ratio of the level of Ki-67 per 1000 circulating lymphocytes (Ki-67 U/1000 lymphocytes).

TABLE 5
Correlation of circulating Ki-67 index
values with various clinical parameters.

| Variable | Valid-N | Spearman-R | t(N − 2) | P |
|---|---|---|---|---|
| HGB (g/dL) | 194 | 0.022 | 03.310 | 0.756 |
| PLT (×10⁶/L) | 194 | 0.165 | 2.325 | 0.021 |
| WBC (×10⁶/L) | 194 | −0.865 | −23.900 | <0.001 |
| Liver enlargement (cm) | 192 | .071 | 0.981 | 0.328 |
| Spleen enlargement (cm) | 187 | −0.194 | −2.684 | 0.008 |
| Lymph node sites- | 190 | −0.299 | −4.292 | <0.001 |
| enlarged | | | | |
| Lymphocytes (%) | 194 | −0.717 | −14.288 | <0.001 |
| B2M (mg/L) | 194 | −0.113 | −1.575 | 0.116 |
| BM Cellularity (%) | 189 | −0.401 | −6.001 | <0.001 |
| BM-Lymphocytes (%) | 194 | −0.525 | −8.567 | <0.001 |
| RAI Stage | 193 | −0.169 | −2.363 | 0.019 |
| Albumin | 193 | −0.067 | −0.929 | 0.354 |
| Creatinine (mg/dL) | 193 | −0.113 | −1.575 | 0.116 |
| IgG (mg/dL) | 184 | 0.009 | 0.125 | 0.900 |
| IgA (mg/dL) | 183 | 0.095 | 1.296 | 0.194 |
| IgM (mg/dlL | 184 | 0.154 | 2.115 | 0.035 |
| Total Protein (g/dL) | 58 | −0.096 | −0.726 | 0.470 |
| CD11/CD22 (%) | 194 | −0.111 | −1.551 | 0.122 |
| CD11C (%) | 194 | −0.152 | −2.140 | 0.033 |
| CD22 (%) | 194 | −0.099 | −1.384 | 0.167 |

Abbreviations:
HGB: hemoglobin;
PLT: platelet count;
WBC: white blood cell count;
B2M: β2 microglobulin;
BM: Bone Marrow;
Ig, immunoglobulin.

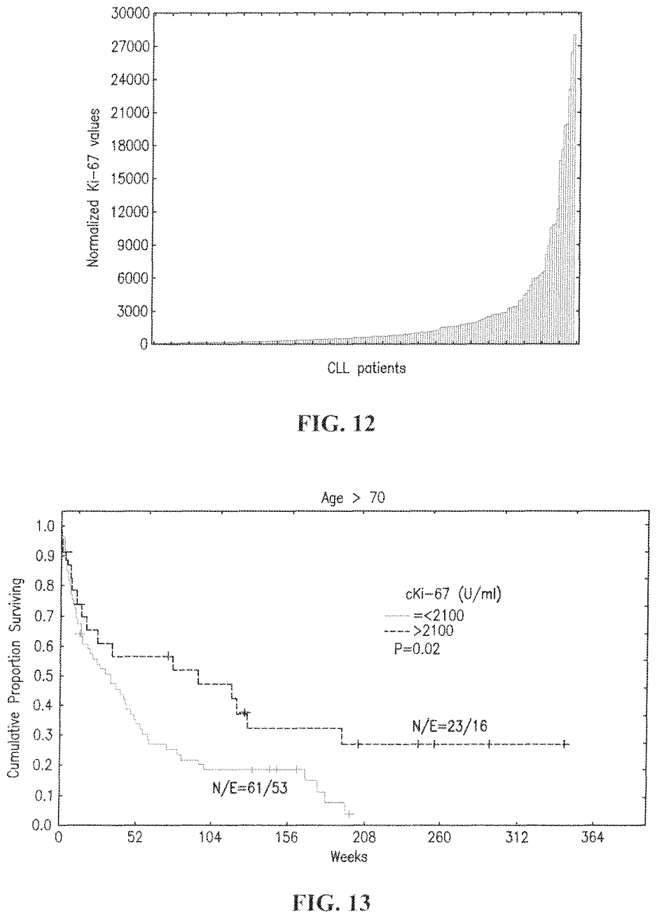

The circulating Ki-67 index values in CLL patients (n=194) were plotted and shown in FIG. 12 which illustrates the range and population profile of the measured Ki-67 index. As shown in Table 4, there was a significant correlation between the circulating Ki-67 index and several hematological markers including white blood cell count, lymphocyte count, percent of bone marrow lymphocytes, RAI staging, spleen enlargement, and number of lymph node sites. However, the circulating Ki-67 index did not correlate with IgVH mutation status (p=0.62) in a Wilcoxon paired test. The Ki-67 index, as a continuous variable, was significantly associate with survival in a Cox regression model (p=0.02) and was a predictor of survival when a cut-off value of 1.20 U/1000 lymphocytes was used (p=0.005; log rank test), wherein patients having a higher Ki-67 index values had shorter survival than those with lower Ki-67 index values (FIG. 15).

The association of the Ki-67 index with survival was independent of the IgVH mutation status (Table 6, Model 1). However, in a multivariate model incorporating the Ki-67 index with B2-microglobulin and IgVH, only the Ki-67 index and B2-microglobulin remained significant predictors of survival (Table 6, Model 2).

TABLE 6
Multivariate Modeling

| | | | | Exponent- | Wald- | |
|---|---|---|---|---|---|---|
| | Beta | S.E.M. | t-value | beta | Statist. | p |
| Model #1 | | | | | | |
| cKi-67 Index | 0.926 | 0.412 | 2.250 | 2.257 | 5.064 | 0.024 |
| IgV_{H} | −1.152 | 0.541 | −2.129 | 0.316 | 4.533 | 0.033 |
| Model #2 | | | | | | |
| cKi-67 Index | 1.012 | 0.415 | 2.44 | 2.751 | 5.954 | 0.015 |
| IgV_{H} | −0.800 | 0.551 | −1.453 | 0.449 | 2.111 | 0.146 |
| B2M | 0.287 | 0.062 | 4.599 | 1.332 | 21.151 | <0.001 |

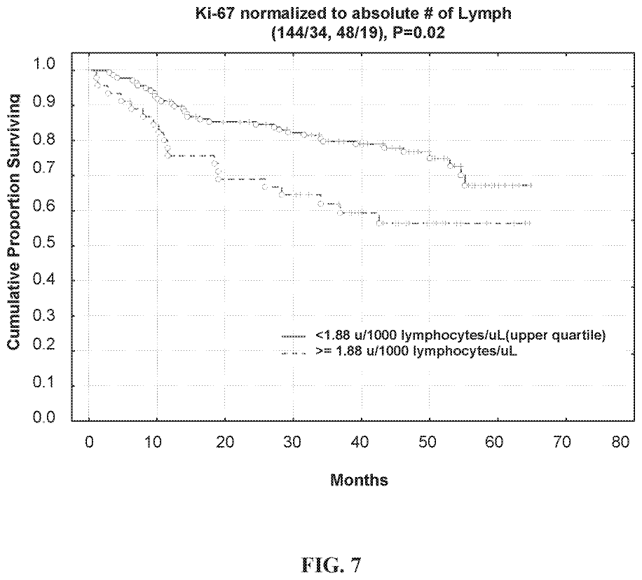

In another experiment, CLL patients were divided into two groups: those with a circulating Ki-67 index value of less than 1.88 U/1000 lymphocytes (upper quartile for survival) and those with a circulating Ki-67 index value grater than or equal to 1.88 U/1000 lymphocytes. Cumulative proportion of CLL patients surviving is plotted against months of survival. CLL patients with circulating Ki-67 index value lower than 1.88 U/1000 lymphocytes/μl plasma had higher survival rate than the CLL patients with circulating Ki-67 index value greater than or equal to 1.88 U/1000 lymphocytes/μl plasma and shown in FIG. 7

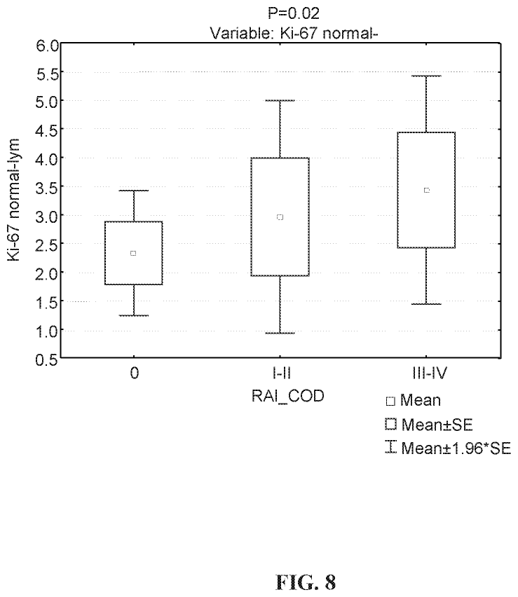

CLL patients were classified based on Rai classification into 5 stages 0, I, II, III, and IV based on increased risk of CLL patients, 0 is the lowest risk and IV has the highest risk. The circulating Ki-67 index value (Ki-67 U/1000 lymphocytes) was plotted against the Rai classifications and indicates a significant prognostic association (FIG. 8). CLL patients with Rai classification of III-IV had the highest relative Ki-67 values while CLL patients with Rai classification of 0 had the lowest relative Ki-67 values. CLL patients with Rai classification of I-II had intermediate Ki-67 values.

### Example 6

### Association of the Ki-67 Index in CLL Patients Undergoing Chemotherapy

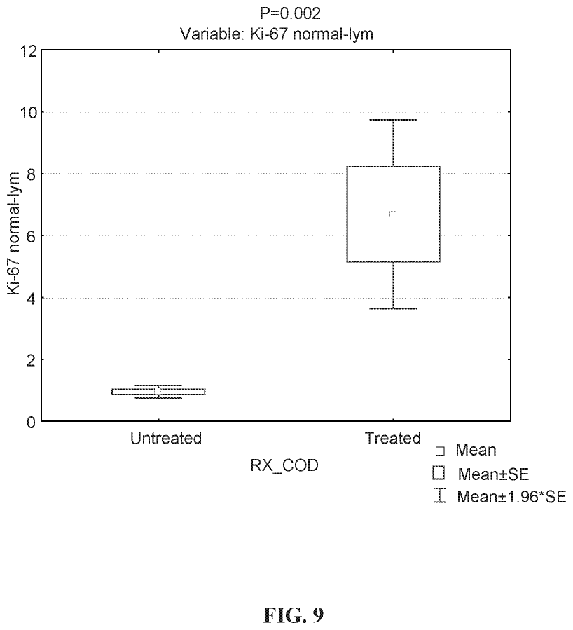

CLL patients were either untreated or treated with Cytoxan, and Fludarabine. Blood samples were collected from CLL patients at the time of initiation of treatment. Some CLL patients in the group had a history of prior treatment, but were off therapy at the time of obtaining blood samples. The circulating Ki-67 index value was obtained and expressed as Ki-67 units per 1000 circulating lymphocytes. The circulating Ki-67 index value of the treated and untreated CLL groups were plotted. FIG. 9 indicates following treatment with Cytoxan, and Fludarabine, CLL patients showed higher levels of circulating Ki-67 index value in plasma as compared to their untreated counterparts.

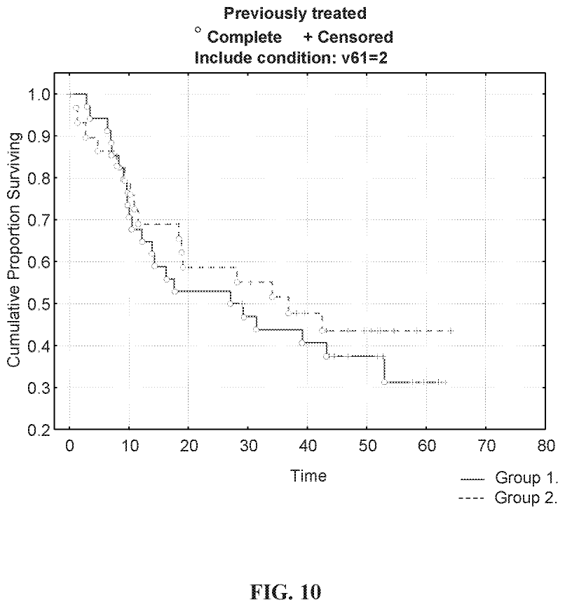

The Ki-67 index (Ki-67 U/1000 lymphocytes) was measured in CLL patients prior to initiating new therapy. Patients were divided into two groups: those with a normalized Ki-67 value of less than 1.88 U/1000 lymphocytes (Group 2) and those with a normalized Ki-67 value grater than or equal to 1.88 units per 1000 lymphocytes (Group 1). Cumulative proportion of CLL patients surviving is plotted against months of survival. As shown in FIG. 10, CLL patients with a circulating Ki-67 index value lower than 1.88 units per 1000 lymphocytes had a higher survival rate than CLL patients with a circulating Ki-67 index value greater than or equal to 1.88 units per 1000 circulating lymphocytes.

### Example 7

### Ki-67 Protein in Patients with Chronic Myeloid Leukemia

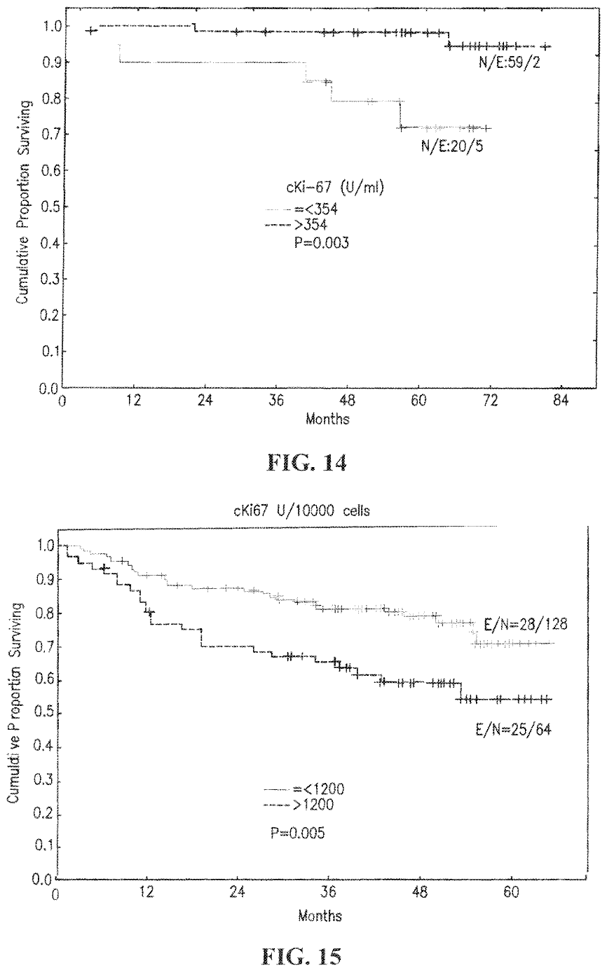

Plasma Ki-67 levels were measured in a study of 81 CML patients in chronic phase and 46 CML patients in accelerated/blast phase. Overall, patients with CML had significantly (P<0.0001) higher levels of Ki-67 in plasma. However, there was no significant difference between accelerated/blast crisis group (median 709.50 U/ml, range: 100-3530.0 U/ml) and the chronic phase group (523 U/ml, range: 73-5857 U/ml) for Ki-67 levels in individual's plasma. Ki-67 levels did not correlate with white cell count or blast count in the chronic phase nor in the accelerated/blast crisis phase. There was no correlation between Ki-67 levels and response to imatinib therapy. However, patients in the chronic phase with higher levels of Ki-67 (>354 U/ml) had significantly longer survival (P=0.003) as shown in FIG. 14. It possible that higher levels of Ki-67 reflect that more stem cells in these patients are in cell cycle and this may make them more susceptible to chemotherapy. Levels of Ki-67 in patients with accelerated/blast phase did not correlate with outcome.

Thus, the data suggests higher proliferation is associated with better survival, most likely confirming that cells in progressing cell cycle may respond better to chemotherapy. This information is important while considering new therapeutic approaches that target cell cycle.

### Example 8

### Association of Plasma Ki-67 with Adult Acute Lymphoblastic Leukemia

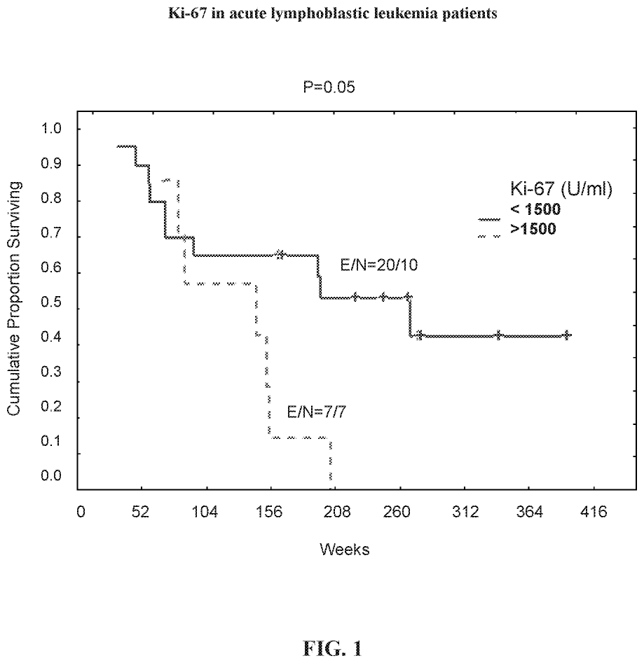

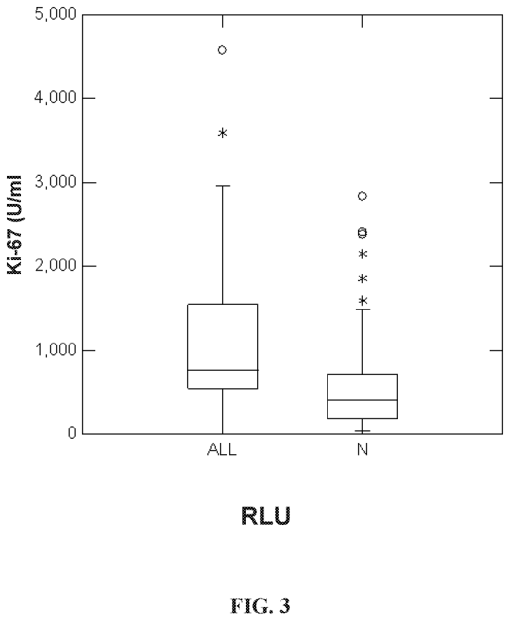

Ki-67 protein was measured using Meso Scale Discovery (MSD) and Immunoblot in the plasma of patients with adult acute lymphoblastic leukemia (ALL) (n=27) and normal patients (n=114). Ki-67 protein levels were significantly higher in patients with ALL (median: 762.10 U/ml; range: 0-4574.03 U/ml) than the normal controls (median: 399.2 U/ml; range: 2830.7 U/ml) and shown in FIG. 3. Patients with higher plasma levels of Ki-67 protein had significantly shorter survival than patients with low levels (FIG. 1). Two of the 27 ALL cases were classified as Burkitt's lymphoma and showed high levels of Ki-67 protein in plasma (median: 999 U/ml; range: 1623 U/ml of plasma). Patients older than 70 years of age with ALL had significantly higher Ki-67 protein levels (P=0.05). These data show that measuring Ki-67 protein levels in plasma have a prognostic value in ALL and also demonstrate that Ki-67 protein can be used as a tumor marker in patients with ALL.

In a second study, the Ki-67 level was determined for a group of 106 newly diagnosed AML patients and 98 control. Patients with AML had significantly higher levels of Ki-67 (median: 1300.74 U/ml, range: 0-6789.0 U/ml) as compared with normal control (median: 339.20 U/ml, range: 0-35.76 U/ml) (P<0.00001). The Ki-67 level did not correlate with white cell count, hemoglobin, platelets, LDH, blast count, age, cytogenetic grouping, or performance status. However, patients with high levels of Ki-67 (above the upper quartile of 2100 U/ml) had significantly longer survival in older patient group (>70) (n=84) (P=0.02) as shown in FIG. 13. In addition patients in the poor cytogenetic group (n=38) with >2100 U/ml also had significantly longer survival (P=0.006).

The contents of the articles, patents, and patent applications, and all other documents and electronically available information mentioned or cited herein, are hereby incorporated by reference in their entirety to the same extent as if each individual publication was specifically and individually indicated to be incorporated by reference.

Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

1-24. (canceled)

25. A method of identifying a circulating Ki-67 index in a subject comprising:
(a) obtaining a blood sample from the subject;
(b) measuring a concentration of lymphocytes in the blood sample from the subject;
(c) contacting a plasma portion of the blood sample with an anti-Ki-67 antibody to form an antibody-antigen complex;
(d) performing an immunoassay with the antibody-antigen complex to determine a concentration of Ki-67 protein in the plasma portion of the blood sample; and
(e) calculating a circulating Ki-67 index by dividing the concentration of Ki-67 protein determined in step (d) by the concentration of lymphocytes measured in step (b), thereby identifying the circulating Ki-67 index in the subject.

26. The method of claim 25, wherein step (c) comprises separating the blood sample from the subject to produce the plasma portion of the blood sample containing less than about 1% (w/w) whole cellular material.

27. The method of claim 25, wherein the anti-Ki-67 antibody is labeled with a detectable label.

28. The method of claim 27, wherein the detectable label is selected from the group consisting of a radioisotope, enzyme, enzyme substrate, luminescent substance, biotin-labeled streptavidin complex, and a predetermined polypeptide epitope recognized by a secondary reporter.

29. The method of claim 25, wherein the anti-Ki-67 antibody is immobilized on a solid support.

30. The method of claim 29, wherein the solid support is selected from the group consisting of a microwell, bead, and microarray.

31. The method of claim 25, wherein the immunoassay is selected from the group consisting of a radioimmunoassay, immunoprecipitation, Western blot, enzyme-linked immunosorbent assay (ELISA), electrochemiluminescence assay, flow cytometry, and a 2-site or sandwich immunoassay.

32. The method of claim 25, wherein the circulating Ki-67 index is expressed as units of Ki-67 per 1000 lymphocytes.

33. The method of claim 32, further comprising comparing the circulating Ki-67 index to a pre-determined cutoff value of about 1.88 U/1000 lymphocytes.

34. The method of claim 25, wherein the Ki-67 protein comprises the amino acid sequence of SEQ ID NO: 1.

35. The method of claim 25, wherein the subject has been diagnosed with or is suspected of having a hematological cancer comprising chronic lymphocytic leukemia (CLL), chronic myeloid leukemia (CML), acute myeloid leukemia (AML), or acute lymphoblastic leukemia (ALL).

36. The method of claim 25, wherein the subject has been diagnosed with or is suspected of having a cancer selected from the group consisting of breast cancer and prostate cancer.

37. A method of identifying a change in Ki-67 protein levels in plasma samples from a subject suspected of or having a leukemia, comprising:
(a) obtaining a first plasma sample from the subject at a first time point and obtaining a second plasma sample from the subject at a second time point, wherein each plasma sample contains less than about 1% (w/w) whole cellular material, and wherein the subject receives chemotherapy between the first time point and the second time point;
(b) contacting each of the first and second plasma samples with an anti-Ki-67 antibody to form a first antibody-antigen complex and a second antibody-antigen complex;
(c) performing an immunoassay on each of the first and second plasma samples to determine a first Ki-67 protein level and a second Ki-67 protein level from the first and second antibody-antigen complexes; and
(d) measuring the first Ki-67 protein level against the second Ki-67 protein level, thereby identifying the change in the Ki-67 protein levels in the plasma samples.

38. The method of claim 37, wherein the immunoassay is selected from a radioimmunoassay, immunoprecipitation, Western blot, enzyme-linked immunosorbent assay (ELISA), electrochemiluminescence assay, flow cytometry, and a 2-site or sandwich immunoassay.

39. The method of claim 37, wherein step (d) further comprises calculating a first Ki-67 index and a second Ki-67 index, wherein each Ki-67 index is a ratio of a respective Ki-67 protein level to an absolute lymphocyte count of the subject at each respective time point.

40. The method of claim 37, wherein the leukemia is CLL, CML, AML, or ALL.

41. The method of claim 37, wherein the chemotherapy comprises Chlorambucil, Cytoxan, or Fludarabine.

42. The method of claim 37, wherein the Ki-67 protein comprises the amino acid sequence of SEQ ID NO: 1.

43. A method of identifying a Ki-67 protein level in a plasma sample from a subject suspected of having a hematological cancer, said method comprising:
(a) obtaining the plasma sample from the subject, wherein the plasma sample contains less than about 1% (w/w) whole cellular material;
(b) contacting the plasma sample with an anti-Ki-67 antibody that is immobilized on a solid support to capture Ki-67 protein from the plasma sample;
(c) contacting the captured Ki-67 protein with a second anti-Ki-67 antibody that is labeled with a detectable label; and
(d) quantifying the Ki-67 protein level in the plasma sample by measuring a signal from the detectable label,
thereby identifying the Ki-67 protein level in the plasma sample.

44. The method of claim 43, wherein the solid support is selected from the group consisting of a microwell, bead, and microarray.

45. The method of claim 43, wherein the detectable label is selected from the group consisting of a radioisotope, enzyme, enzyme substrate, luminescent substance, biotin-labeled streptavidin complex, and a predetermined polypeptide epitope recognized by a secondary reporter.

46. The method of claim 43, wherein the hematological cancer is selected from the group consisting of CLL, CML, AML, ALL, and non-Hodgkin's lymphoma (NHL).

47. The method of claim 43, further comprising comparing the Ki-67 protein level to a pre-determined reference level, wherein the pre-determined reference level is selected from the group consisting of about 354 U/mL, about 900 U/mL, about 1500 U/mL, and about 2100 U/mL.

48. The method of claim 43, further comprising measuring a level of β2-microglobulin in the plasma sample.

49. The method of claim 43, wherein the quantifying of step (d) is performed by radioimmunoassay, immunoprecipitation, Western blot, enzyme-linked immunosorbent assay (ELISA), electrochemiluminescence assay, flow cytometry, or a 2-site or sandwich immunoassay.

50. A method of identifying a circulating Ki-67 index value in a subject diagnosed with leukemia, said method comprising:
(a) obtaining a plasma sample from the subject, wherein the plasma sample contains less than about 1% (w/w) whole cellular material;
(b) measuring a concentration of lymphocytes in a blood sample from the subject;
(c) contacting the plasma sample with an anti-Ki-67 antibody;
(d) performing an immunoassay to determine a concentration of Ki-67 protein in the plasma sample;
(e) calculating a circulating Ki-67 index by dividing the concentration of Ki-67 protein determined in step (d) by the concentration of lymphocytes measured in step (b); and
(f) comparing the circulating Ki-67 index to a pre-determined cutoff value, thereby identifying the circulating Ki-67 index value in the subject.

51. The method of claim 50, wherein the circulating Ki-67 index value is expressed as units of Ki-67 per 1000 lymphocytes.

52. The method of claim 51, wherein the pre-determined cutoff value is about 1.5 to about 2.25 U/1000 lymphocytes.

53. The method of claim 52, wherein the pre-determined cutoff value is about 1.88 U/1000 lymphocytes.

54. The method of claim 51, wherein the pre-determined cutoff value is about 1.20 U/1000 lymphocytes.

55. The method of claim 50, wherein the immunoassay of step (d) is an electrochemiluminescence immunoassay.

56. The method of claim 50, wherein the leukemia is selected from the group consisting of CLL, CML, AML, ALL, and acute undifferentiated leukemia (AUL).
